(19) United States
(12) Patent Application Publication
(10) Pub. No.: US 2007/0172886 A1
(43) Pub. Date: Jul. 26, 2007

(54) Reaction module for biological analysis

(76) Inventors: Bruno Colin, March L'Etoile (FR); Frederic Paulhet, Bron (FR)

Correspondence Address: OLIFF & BERRIDGE, PLC, P.O. BOX 19928, ALEXANDRIA, VA, 22320, US

(21) Appl. No.: 10/553,440

(22) PCT Filed: May 6, 2004
(86) PCT No.: PCT/FR04/50181

§ 371 Date: Jul. 13, 2006

(30) Foreign Application Priority Data
May 7, 2003 (FR) FR030554

(51) Int. Cl. G01N 33/53; C12M 3/00

(52) U.S. Cl. 435/7.1; 435/287.2

(57) ABSTRACT

The present invention relates to a reaction module for biological analysis comprising the injection of biological fluid and/or of reagent allowing a determined biological reaction comprising at least one means for controlling the quantity of biological fluid and/or of reagent injected.

The present invention relates to automated apparatus for carrying out immunological tests. The invention relates particularly to a means for controlling these tests.

Medical biology plays a major role in public health, whether in the diagnosis of disease, the management of patients and their treatment. Automated instruments intended for medical analyses and for quality control of products in the food and cosmetics industries, and the like are in this regard commonly used in the laboratory. A number of automated diagnostic apparatus exists on the market which make it possible in particular, by immunological assays such as an ELISA test, the identification of pathogenic agents responsible for numerous diseases. These automated devices for immunological analysis generally use automated successive steps of injection/aspiration of the biological sample which it is desired to analyse and of reagents for the detection of one or more given pathogenic agents. These automated devices allow in particular the development of an ELISA (Enzyme Linked ImmunoSorbent Assay) type sandwich test which is rapid and for a large number of samples. Thus, as a guide, the first step may consist in coating, onto the walls of a solid support recognition antibodies which are specific for a target antigen of a given pathogenic agent which it is desired to identify in a biological sample from a patient. The second step then consists in bringing the biological sample from the patient into contact with the said recognition antibodies. If the sample from the patient contains the target antigen, a complex forms between the target antigen and the recognition antibody. The target antigens attached to the recognition antibodies are then brought into contact with labelled, detection antibodies which make it possible to reveal the presence of the recognition antibody-target antigen-detection antibody sandwich. Such tests are well known to persons skilled in the art.

By way of example of automated apparatus for immunological assays, there may be mentioned in particular the VIDAS® apparatus which is a multiparametric automated device for immunoanalyses. This apparatus is composed of an analytic module which automatically manages all the steps of the analysis up to the complete editing of the results. The principle of the VIDAS® test is based on the use of a tip and of a reaction strip:
the tip is the solid phase for the reaction and comprises the recognition antibodies coated onto its wall. It is for single use and serves as sampling needle. It is subjected during the test to injection/aspiration steps in order to successively bring the multiple test reagents into contact.
the reaction strip contains all the ready-to-use reagents distributed in the various wells of the strip, and in particular the detection antibodies. A well also allows deposition of the sample which it is desired to analyse.

At each step of the immunoanalysis, the tip aspirates and discharges several times the reagents contained in the different wells of the strip up to the final step of the analysis. The last well of the strip is the reading cell where the final intensity of the reaction is measured by fluorescence.

In general, these automated apparatus require a control for good operation which has to be carried out regularly in order to avoid any risk of false-positives, that is to say indicate to the patient that they are ill when they are not, but also of false-negatives, that is to say indicate to the patient that they are not ill when they are. Such false-negatives may be observed in particular when the injection of various reagents does not occur, or when the reagent volume is not the correct volume. In addition to maintenance which is carried out regularly, which makes it possible to avoid such false-negatives, this control may be performed by weighing the individual reaction strips before and after the test, but that involves a cumbersome protocol for the user. The control may also be carried out by pressure sensors, located inside the automated device, in order to detect if the sample or the reagent has indeed been injected. This however increases the cost of manufacturing the automated devices.

The present invention proposes to solve all the disadvantages of the state of the art by improving the current systems for controlling automated devices for biological assays and by offering a very simple and rapid control system allowing visual interpretation of the results, which is inexpensive, without requiring complete calibration of the automated device for biological assays.

Before proceeding further, a few definitions are given in order to facilitate understanding of the disclosure of the invention.

The expression reaction module is understood to mean any device capable of being inserted into an automated device for biological assays in order to carry out a biological reaction. As a guide, this reaction module may be a reaction strip comprising several reaction wells as used in the Vidas® automated devices, but may also be a 96-well microplate, or any other container used by persons skilled in the art for carrying out immunological tests.

The expression biological fluid is understood to mean any fluid in which it is desired to detect the presence of a given antigen (or antibody). This fluid may thus be a clinical blood, urine, saliva or plasma sample, and the like. This fluid may also be a food sample consisting of water or drinks in which it is desired to determine the presence of an organism (bacteria, parasites, viruses and the like).

Preferably, the biological fluid is a clinical blood, urine or plasma sample.

The expression reagent is understood to mean any chemical solution necessary for developing an immunological test. Such a solution may comprise in particular recognition antibodies, detection antibodies, but also washing solutions and the like. The expression biological reaction is understood to mean any reaction capable of detecting the presence of a given antigen (or antibody). Preferably, this biological reaction is an antigen-antibody recognition reaction.

The expression control means is understood to mean a means which makes it possible to detect the presence of false-negatives or of false-positives in a test. This control means may be in particular absorbent paper comprising a dehydrated dye capable of diffusing and of creating a calorimetric signal when it is in the presence of a biological fluid and/or of a reagent. This control means can allow a quantitative control of the required volume of biological fluid and/or reagent during the immunological test, and/or a qualitative control.

To this effect, the present invention relates to a reaction module comprising at least one reaction well for biological analysis comprising the injection of biological fluid and/or of reagent allowing a determined biological reaction and at least one means for controlling the quantity of biological fluid and/or of reagent injected, characterized in that the control means is a calibrated colorimetric strip.

According to a preferred embodiment of the invention, the module comprises a graduated scale along the strip for a visual determination of the volume. This calibrated colorimetric strip thus allows a quantitative control of the biological analysis through the verification of the injected volume of biological fluid and/or of reagent during each step of the biological analysis.

The invention also relates to a module as defined above, characterized in that the control means is a calorimetric pellet. In this case, the colorimetric pellet allows a qualitative control of the biological analysis by the verification of the injection of biological fluid and/or of reagent during each step of the biological analysis.

The accompanying figures are given by way of explanatory example and are not at all limiting. They will allow better understanding of the invention.

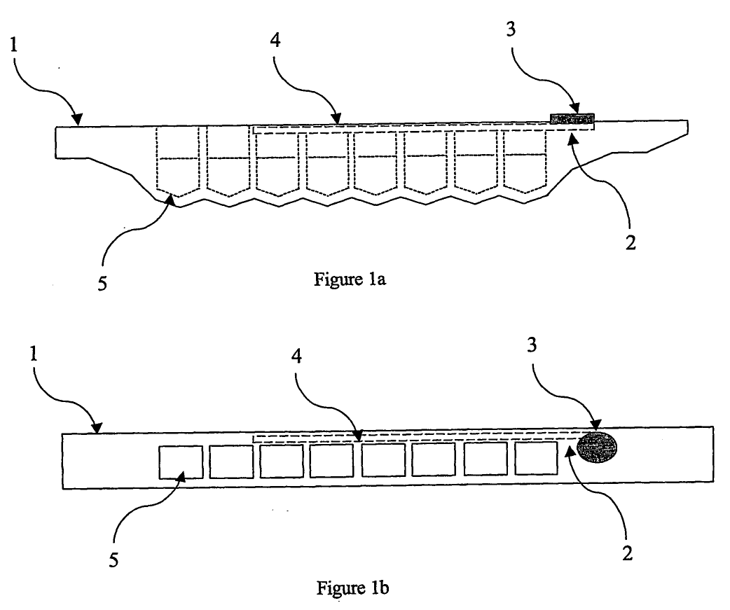

FIG. 1 represents a first embodiment of the invention. FIG. 1a represents a side view of a reaction module which is a reaction strip (1) comprising a control means (2) according to the invention. This control means is an absorbent paper strip comprising a calibrated volumetric scale (4). The injection of biological fluid and/or of reagent is carried out through an orifice (3), and the fluid diffuses along the volumetric scale. Preferably, the calibrated volumetric scale (4) is integrated into the strip so that the user is not in direct contact with the biological fluid and/or the reagent which is absorbed by the control means (2). This is important especially when the biological fluid is likely to be contaminated. This strip comprises 8 reaction wells (5). FIG. 1b represents a top view of a reaction strip (1) comprising the control means as defined above.

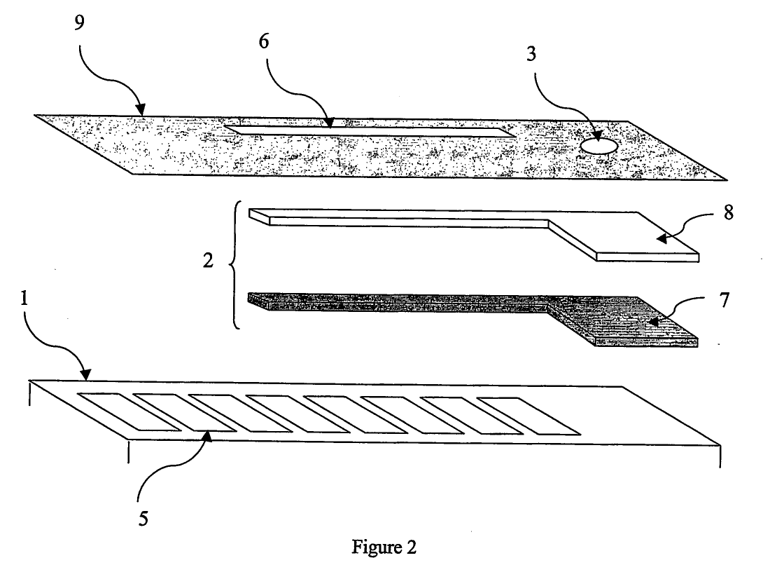

FIG. 2 represents in more detail the control means (2) presented in FIG. 1. Various layers which are superposed on the reaction strip (1) comprising the wells (5) are successively distinguishable in this case. A first absorbent layer (7) is deposited on the strip (1). This first layer comprises a dried dye. A second layer (8) made of absorbent paper but comprising no dye is placed above. These first and second layers make it possible to obtain a simple and inexpensive control means: when a fluid is in contact with the second layer (8), it diffuses across up to the first layer (7), rehydrating the dye which diffuses in turn inside the second layer (8). When the second layer (8) is stained, that means, for the user, that an injection of fluid has occurred. Finally, a final layer, which is a protective means (9) such as a plastic film, makes it possible to isolate the control means (2). An orifice (3) for the deposition of fluid and a reading window (6) are distinguishable in this case.

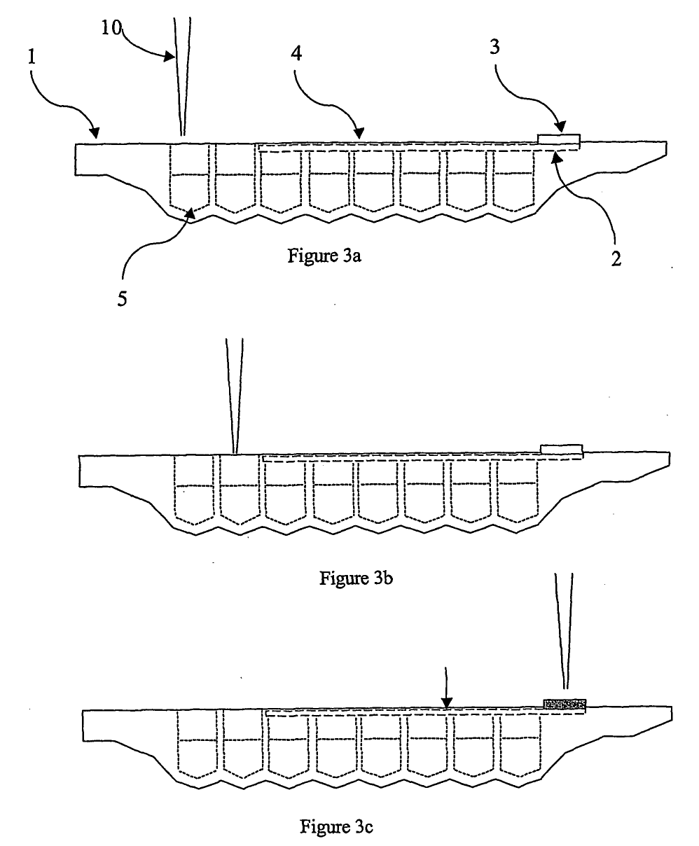

FIG. 3 represents various successive steps performed during an immuno-logical test. FIG. 3a represents the first step of an immunological test such as a Vidas® test in which a reaction tip (10) aspirates a determined volume of biological fluid into the first well of the strip. This reaction tip (10) comprises, on its wall, recognition antibodies which form an antigen-antibody complex with the target antigens of the biological fluid. FIG. 3b represents the second step of the test which consists in aspirating and discharging a reagent such as in particular a washing fluid contained in a second well in order to remove the target antigens which would have been poorly attached to the recognition antibodies. For that, the tip (10) automatically passes from the first well of the strip to the second well. The entire test progresses through successive steps of aspiration/injection by the tip in contact with the various reagents of the various wells. These steps are well known to persons skilled in the art. At the end of the test, and as represented in FIG. 3c, a control step is performed in order to determine if the aspiration/injection steps were performed correctly. For that, the automated device collects with the tip (10) a determined volume of fluid (which may be a biological fluid, a reagent or a simple aqueous solution) from the last well of the reaction strip and deposits this volume onto the control means (2). The fluid then diffuses inside the second layer as presented in FIG. 2 until it reaches the first layer comprising a dye. This rehydrated dye then diffuses until it reaches the surface of the second layer of dye. When fluid is injected, the user can then easily visualize the dye which diffuses along the calibrated volumetric scale, the diffusion being proportional to the volume injected. This thus allows easy quantitative control of the test previously carried out.

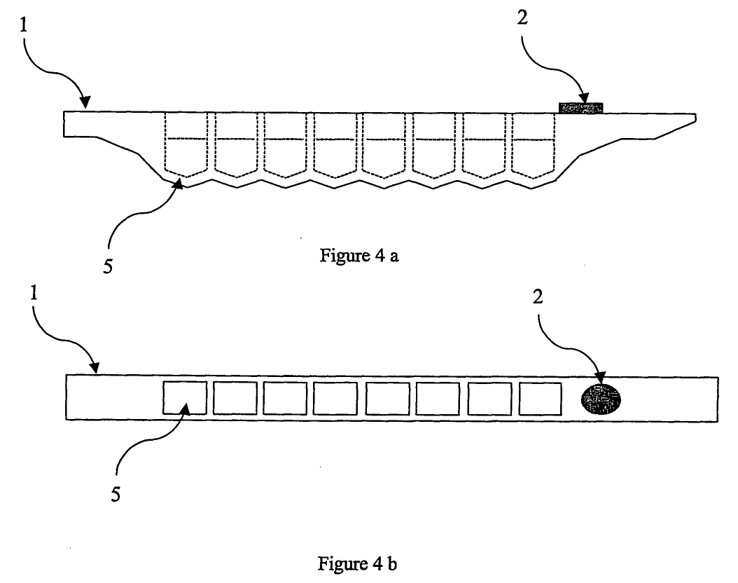

FIG. 4 presents another control means according to the invention. The control means is in this case a colorimetric pellet which allows a qualitative control of the aspiration/injection steps performed during the test.

### REFERENCES

1. reaction strip
2. control means
3. orifice for deposition of fluid
4. graduated volumetric scale
5. reaction well
6. reading window
7. first absorbent layer comprising a dye
8. second absorbent layer
9. protective means
10. reaction tip

1. Reaction module comprising at least one reaction well for biological analysis for the injection of biological fluid and/or of reagent allowing a determined biological reaction and comprising at least one means for controlling the quantity of biological fluid and/or of reagent injected, characterized in that the control means is a calibrated colorimetric strip.

2. Module according to claim 2, characterized in that it comprises a graduated scale along the strip for a visual determination of the volume.

3. Reaction module comprising at least one reaction well for biological analysis for the injection of biological fluid and/or of reagent allowing a determined biological reaction and comprising at least one means for controlling the quantity of biological fluid and/or of reagent injected, characterized in that the control means is a calorimetric pellet.
